(19) United States
(12) Patent Application Publication
(10) Pub. No.: US 2009/0069671 A1
(43) Pub. Date: Mar. 12, 2009

(54) Electric Motor Tracking System and Method

(75) Inventor: Peter Traneus Anderson, Andover, MA (US)

Correspondence Address: GE HEALTHCARE;c/o FLETCHER YODER, PC, P.O. BOX 692289, HOUSTON, TX, 77269-2289, US

(73) Assignee: General Electric Company, Schenactady, NY (US)

(21) Appl. No.: 11/852,597

(22) Filed: Sep. 10, 2007

(51) Int. Cl. A61B 5/06

(52) U.S. Cl. 600/424; 324/260

(57) ABSTRACT

Provided is a tracking system, including a motor coupled to a medical instrument and configured to generate at least one magnetic field, and at least one receiver coil configured to sense the magnetic field. Also provided is a method of tracking, including rotating a rotor of a motor, wherein the rotor comprises a permanent magnet that generates a rotating magnetic field when the rotor is rotated, sensing the rotating magnetic field with at least one receiver, transmitting to a processor a signal indicative of the rotating magnetic field, and processing the signal to determine a position of the motor. Further provided is a method of tracking, comprising energizing a stator coil of a motor to generate at least one magnetic field, sensing the at least one magnetic field with at least one receiver, transmitting to a processor a signal indicative of the at least one magnetic field, and processing the signal to determine a position of the motor.

### BACKGROUND

This disclosure generally relates to tracking systems that employ magnetic fields to determine the position and orientation of an object, such as systems used for tracking instruments and devices during surgical interventions and other medical procedures. More particularly, this disclosure relates to a system and method that utilizes at least one magnetic field emitted from an electric motor for tracking.

Tracking systems have been used in various industries and applications to provide positional information relating to various objects and devices. For example, electromagnetic tracking is useful in aviation applications, motion sensing applications, medical applications, and the like. In medical applications, tracking systems are employed to provide information to an operator (e.g., a clinician) that assist in locating a medical instrument or device that is not the line of sight of the clinician (e.g., disposed internal to a patient). The information generally includes an image having a base image displayed on a monitor, and a visual indication of the instrument's position. For instance, the image may include a visualization of the patient's anatomy with an overlay (e.g., icon) that represents the location of the instrument relative to the patient. Typically, the displayed image is continuously updated to reflect the current position of the device. The base image of the patient's anatomy maybe generated prior to, or during the medical procedure. For example, any suitable medical imaging technique, such as X-ray, computed tomography (CT), magnetic resonance imaging (MRI), positron emission tomography (PET), and ultrasound, may be utilized to generate the base image. Accordingly, the combination of the base image and the representation of the tracked instrument provide positioning information that enables a medical practitioner to manipulate the instrument to a desired location and position and to associate gathered information to a precise location.

To track a device, tracking systems utilize a variety of methods, including magnetic field generation and detection. In a system utilizing magnetic field generation and detection, at least one magnetic field is provided from a magnetic field source (e.g., transmitter), and the magnetic field is sensed by one or more sensors (e.g., receivers). In some systems, the transmitter includes a permanent magnetic, an electromagnet, or a combination thereof. Further, the receiver generally includes a sensing device, such as a coil of conductive material that is responsive to a magnetic field. For example, when a receiver coil is exposed to a magnetic field, a current and voltage indicative of the strength of the magnetic field is driven across the coil. Thus, based on the sensed magnetic field strength, processing can determine the position of the transmitter and receiver relative to one another. For example, processing of the signal may enable a determination of the mutual inductance between each of the transmitters and the receivers, and employs the ratios of the mutual inductance to resolve the positions of the transmitter and the receiver relative to one another.

Further, the tracking system may employ multiple transmitters and/or receivers that enable processing to precisely resolve position and orientation of the transmitters and receivers (e.g., the X, Y and Z coordinates, as well as the roll, pitch and yaw angles). In other words, multiple magnetic fields and receivers enable tracking to resolve a plurality of degrees of freedom. For example, the transmitter may be employed to provide multiple magnetic fields, and/or the system may include multiple receivers. Accordingly, the plurality measurements sensed between the transmitters and receivers can be employed to triangulate a position and/or orientation of the transmitters and the receivers relative to one another.

In medical tracking applications, the space for housing tracking components (e.g., transmitters and receivers) may be extremely limited. For example, catheters that are threaded into the blood vessels of the patient often have an outer diameter of about 1 mm. Thus, devices disposed in a tip of the catheter, such as tracking transmitters, receivers, ultrasonic transducers, motors, and the like, are limited to an extremely confined space (e.g., less than about 1 mm in diameter).

Accordingly, there is a desire to provide a tracking system wherein the tracking components can be disposed within a limited volume. Further, there is a desire to limit the number and complexity of the tracking system components.

### BRIEF DESCRIPTION

In accordance with an aspect, provided is a tracking system, including a motor coupled to a medical instrument and configured to generate at least one magnetic field, and at least one receiver coil configured to sense the magnetic field.

In accordance with another aspect, provided is a method of tracking, including rotating the rotor of a motor, wherein the rotor comprises a permanent magnet that generates a rotating magnetic field when the rotor is rotated, sensing the rotating magnetic field with at least one receiver, transmitting to a processor a signal indicative of the rotating magnetic field, and processing the signal to determine a position of the motor.

In accordance with yet another aspect, provided is a method of tracking, comprising energizing a stator coil of a motor to generate at least one magnetic field, sensing the at least one magnetic field with at least one receiver, transmitting to a processor a signal indicative of the at least one magnetic field, and processing the signal to determine a position of the motor.

### DRAWINGS

These and other features, aspects, and advantages of the present invention will become better understood when the following detailed description is read with reference to the accompanying drawings in which like characters represent like parts throughout the drawings, wherein:

FIG. 1 illustrates an exemplary tracking system implementing certain aspects of the present technique;

FIG. 2 illustrates an embodiment of a catheter in accordance with an aspect of the present technique;

FIG. 3 illustrates a block diagram of an embodiment of a motor in accordance with an aspect of the present technique;

FIG. 4 illustrates a flowchart of a method of tracking in accordance with an aspect of the present technique; and

FIG. 5 illustrates a flowchart of another method of tracking in accordance with an aspect of the present technique.

### DETAILED DESCRIPTION

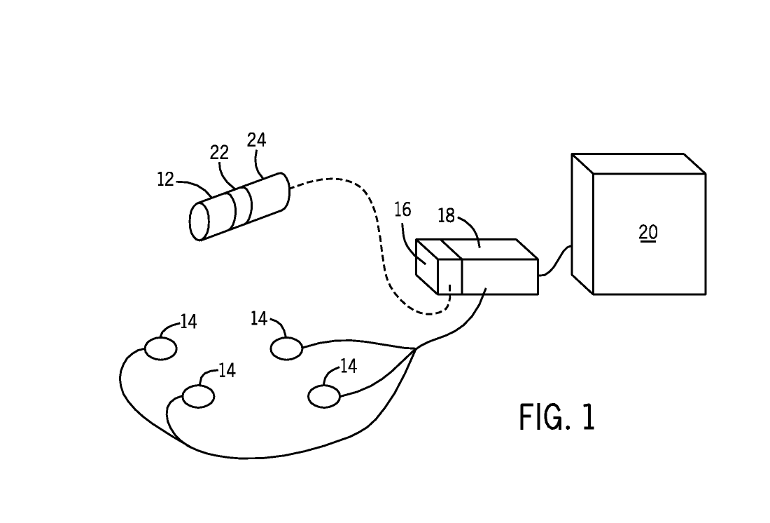

FIG. 1 illustrates a tracking system 10 in accordance with an embodiment of the present technique. The tracking system 10 includes a transmitter 12, at least one receiver 14, a drive unit 16, a processor 18, a user interface 20, a current source 22 and an instrument 24.

The transmitter 12 includes a magnetic field source that can be employed to generate a magnetic field. Accordingly, the magnetic field may be of sufficient magnitude to be sensed by a complementary device, such as the at least one receiver 14. In certain embodiments, the transmitter 12 includes a permanent magnet. For example, in one embodiment, the transmitter 12 includes a rare earth magnet formed from iron, cobalt, nickel, or the like, and having a magnetic moment. Further, in certain embodiments, the transmitter 12 includes a magnetic dipole having a magnitude moment vector along its axis. As will be appreciated, permanent magnets provide a generally constant magnetic field without the need for a drive current or other power source. Thus, an embodiment of the transmitter 12 including a permanent magnet may not require a wired or other electrical connection to other components of the system (e.g., the drive unit 16 or current source 22). In certain embodiments, multiple magnets may be employed to provide a plurality of magnetic fields. In other words, the transmitter 12 may include multiple magnets positioned relative to one another to provide a resulting magnetic field that can be sensed by the receiver 14.

In other embodiments, the transmitter 12 includes an electromagnet that generates the desired magnetic field. In one embodiment, the transmitter 12 includes a single dipole coil. For example, the transmitter 12 may include a single dipole coil that is about 8 mm long and about 1.7 mm in diameter, with 7700 turns of American Wire Gauge (AWG) wire formed around a ferromagnetic core that is about 8 mm long and about 0.5 mm in diameter. When a current is provided across the single dipole coil, a single magnetic field is generated with a magnitude moment vector generally normal to the coil (e.g., along its axis) and having a frequency that is approximately the same as the frequency of the current driving the coil. For example, the transmitters 12 in electromagnetic tracking systems generally are supplied with sine wave current waveforms with frequencies between about 8 kHz and about 40 kHz and, thus, generate magnetic fields with frequencies between about 8 kHz and about 40 kHz.

Further, it may be desirable that the transmitter 12 generates a plurality of magnetic fields and, thus, in one embodiment, the transmitter 12 includes a plurality (e.g., more than one) of the coils. For example, the transmitter 12 may be formed from three co-located orthogonal quasi-dipole coils (i.e., a coil-trio). When a coil-trio is energized, each coil generates a magnetic field. As a result, a coil-trio may be employed to generate three magnetic fields having magnitude vectors that are co-located and orthogonal to one another.

In certain embodiments, the transmitter 12 includes a wireless electromagnet configuration. In other words, the transmitter 12 includes an electromagnet that does not have a wired connection to various components of the tracking system 10 (e.g., the drive unit 16 and the processor 18). As discussed in further detail below, the transmitter 12 may include only the integral current source 22 (e.g., oscillator) and processing may be employed specifically for tracking the transmitter 12 in a wireless configuration.

Complementary to the transmitter 12, the system 10 includes at least one receiver 14. The at least one receiver 14 is configured to sense (i.e., receive) the magnetic field(s) generated by each coil of the transmitter 12. For instance, when a current is applied to at least one coil of the transmitter 12, the magnetic field generated by the transmitter 12 may induce a current and voltage into a coil of the at least one receiver 14. Generally, the induced voltage is indicative of the mutual inductance between the transmitter coil and the receiver coil. Thus, the current and voltage induced across the coil of each of the at least one receiver 14 may be sensed and processed to determine the mutual inductance (Lm) between the transmitter 12 and at least one receiver 14. As is discussed in further detail below with regard to processing, the mutual inductance may be associated with the distance between the coil of the transmitter 12 and the coil of the receiver 14.

Similar to the transmitter 12, the at least one receiver 14 may employ a single dipole coil or multiple coils (e.g., a coil trio). For example, the system 10 may include an electromagnetic tracking system configured with industry-standard coil architecture (ISCA). ISCA type coils are defined as three approximately collocated, approximately orthogonal, and approximately dipole coils. For example, an ISCA configuration includes a three-axis dipole coil transmitter 12 and at least one receiver 14 including a three-axis dipole coil. In such a configuration, the coils of the transmitter 12 and the coils of the at least one receiver 14 are configured such that the three coils exhibit the same effective area, are oriented orthogonally one another, and are centered at the same point. Using this configuration, nine parameter measurements may be obtained (e.g., a measurement between each coil of the transmitter 12 and each coil of the at least one receiver 14). From the nine parameter measurements, processing can determine position and orientation information for each coil of the transmitter 12 with respect to each coil of the at least one receiver 14. If either of the transmitter 12 or receivers 14 is in a known position, processing may also resolve position and orientation relative to the known position.

As mentioned previously, the system 10 may further include a drive unit 16. In accordance with certain implementations of the present technique, the drive unit 16 may be configured to provide a drive current (via a cable) to each coil of the transmitter 12. By way of example, a drive current may be supplied by the drive unit 16 to energize a coil of a transmitter 12 and, thereby, generate a magnetic field that is sensed by the at least one receiver 14. The drive current may include a periodic waveform with a given frequency (e.g., a sine wave). In turn, the current across the coil of the transmitter 12 will generate a magnetic field at the same frequency as the drive current. For example, the transmitters 12 of electromagnetic tracking systems are generally supplied with sinewave current waveforms having frequencies between about 8 kHz and about 40 kHz and, thus, generate magnetic fields having frequencies between about 8 kHz and about 40 kHz. Further, in the illustrated embodiment, the drive unit 16 is integral to the processor 18; however, in other embodiments, the drive unit 16 may include a unit that is separate from the processor 18. For example, the drive unit 16 may include a current source, such as a battery and an oscillator that is integral to an instrument and/or the transmitter 12.

The illustrated system 10 also includes a processor 18. The processor 18 may include, for example, a digital signal processor, a CPU, or the like. In the illustrated embodiment, signals indicative of the magnetic fields sensed by the at least one receiver 14 are output to the processor 18 via a cable. Accordingly, the processor 18 may process the signals to track the orientation and position of the transmitter 12. For example, the at least one receiver 14 produces output signals that are indicative of the mutual inductance between the transmitter 12 and the at least one receiver 14, and the processor 18 may employ the mutual inductance measurements to triangulate the position of the transmitter 12. When the magnetic fields each include different frequencies, processing may include selectively filtering each magnetic field out of the signal. For example, when driving a transmitter (such as transmitter 12) having a single dipole coil, a single drive current of a given frequency may be sufficient to identify the magnetic field. This is true because only a single transmitting coil is generating a magnetic field. However, when the transmitter 12 (e.g., a coil trio) generates multiple magnetic fields, each of the at least one receiver 14 may sense the multiple magnetic fields simultaneously. As a result a single combined signal from the at least one receiver 14 is transmitted to the processor 18. To enable subsequent processing to identify each of the magnetic fields from the combined signal, the frequency of each of the generated magnetic fields is varied. Due the variations in frequency, processing can isolate the signals between each respective transmitter 12 and each coil of the at least one receiver 14 and, thereby, determine the relative position and/or orientation of each of the coils. For example, if each coil of the transmitter 12 is provided a current waveform of a different frequency, processing may identify each magnetic field.

In an embodiment including a wired transmitter, the transmitter 12 may be electrically coupled to the processor 18 and, thus, coupled to the at least one receiver 14. Accordingly, in a wired configuration, the measured phases of the waveforms driving each coil of the transmitter 12 may be known. For example, the source of the drive current waveforms (e.g., the drive unit 16) may be embedded in the processor 18. Therefore, the processor 18 may “know” the phase that is driving the transmitter 12 and can, thus, parse out each signal indicative of the given phases and frequencies from the combined signal sensed the at least one receiver 14. With each of the phases identified and associated with each coil of the transmitter 12, the processor 18 may implement any suitable algorithms to establish the position and/or orientation of the transmitter 12 relative to the at least one receiver 14. An embodiment including a wireless transmitter 12 may not have “known” phases of the current waveforms. For example, the wireless transmitter 12 may be driven by the independent current source 22 that is coupled to the transmitter 12. The current source 22 may include an oscillator, for example. In one configuration, the current source 22 may generate each of the current waveforms independent from the processor 18 (i.e., the processor 18 does not have feedback or control relating to the phase of the two currents). For example, the transmitter 12 may be a standalone device having a current source 22 that is generating magnetic fields independent of the processor 18. Thus, the processor 18 must incorporate additional considerations in processing to resolve the position and orientation of the transmitter 12 (e.g., identify the phases of the current waveforms generated across at least one coil of the transmitter 12).

Methods and techniques for processing the sensed signals to determine the relative position and orientation of the transmitter 12 and the at least on receiver 14 may be found in U.S. Pat. No. 7,158,754, entitled “Electromagnetic Tracking System and Method Using a Single-Coil Transmitter,” issued Jan. 2, 2007 and filed on Jan. 6, 2005, with inventor Peter Anderson, which is herein incorporated by reference.

The illustrated system 10 also includes a user interface 20. For example, the system 10 may include a monitor to display the determined position and orientation of a tracked object. As will be appreciated, the user interface 20 may include additional devices to facilitate the exchange of data between the tracking system 10 and the user. For example, the user interface 20 may include a keyboard, mouse, printers or other peripherals. While the processor 18 and the user interface 20 may be separate devices, in certain embodiments, the processor 18 and the user interface 20 may be provided as a single unit.

Further, in the illustrated tracking system 10, the transmitter 12 is coupled to an instrument 24. Accordingly, the tracking system 10 may be employed to track the position of the instrument 24 relative to the at least one receiver 14, as discussed above. The instrument 24 may include a catheter, a drill, a guide wire, an endoscope, a laparoscope, a biopsy needle, an ablation device or other similar devices.

Although the illustrated tracking system 10 may be employed to provide tracking in a variety of medical and other procedures, the ability to employ the tracking system 10 in certain environments may be limited by the physical space available for the tracking components. For example, in medical tracking applications, the space for housing tracking components (e.g., the transmitter 12 and at least one receiver 14) may be extremely limited. Catheters that are threaded into the blood vessels of a patient often have an outer diameter of about 1 mm. Thus, devices disposed in a tip of the catheter, such as tracking transmitters, receivers, ultrasonic transducers, motors, and the like, are limited to an extremely confined space (e.g., less than about 1 mm in diameter). Further, it may be desirable that the number and complexity of components be decreased. The embodiments discussed below employ a motor device acting as a transmitter for tracking purposes. For example, the following embodiments enable tracking of the motor via the magnetic and electromagnetic fields generated by the motor disposed internal to the catheter. As will be appreciated, the embodiments discussed below may be employed in other similar systems employing an electric motor having similar magnetic characteristics.

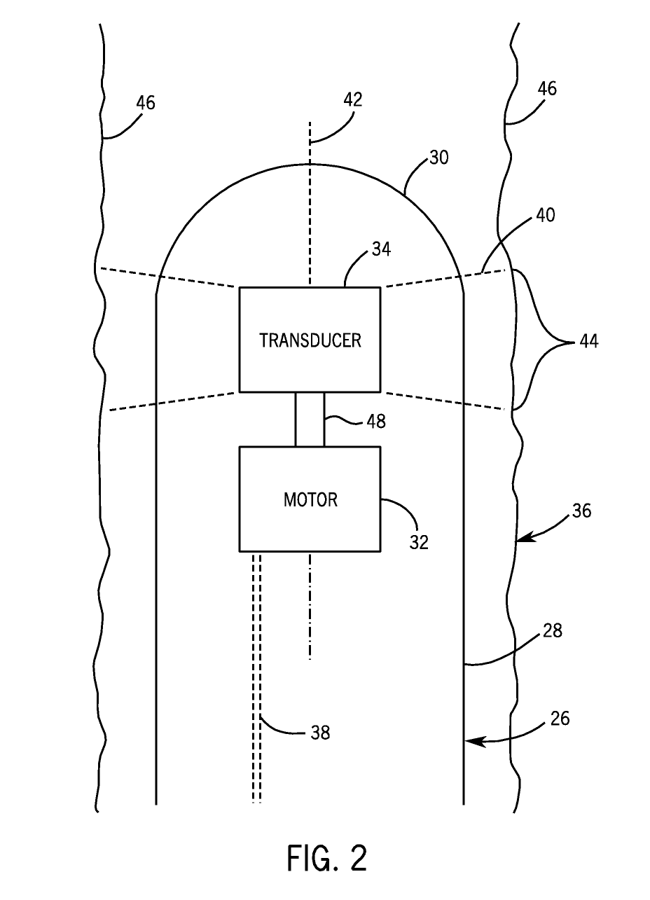

FIG. 2 illustrates an embodiment of a catheter 26 in accordance with aspects of the present technique. In the illustrated embodiment, the catheter 26 includes a body 28, a tip 30, a motor 32, and a transducer 34. The catheter 26 is disposed internal to a passage 36. The passage 36 may include blood vessels (e.g., a vein or an artery), or other cavity of a patient, for instance.

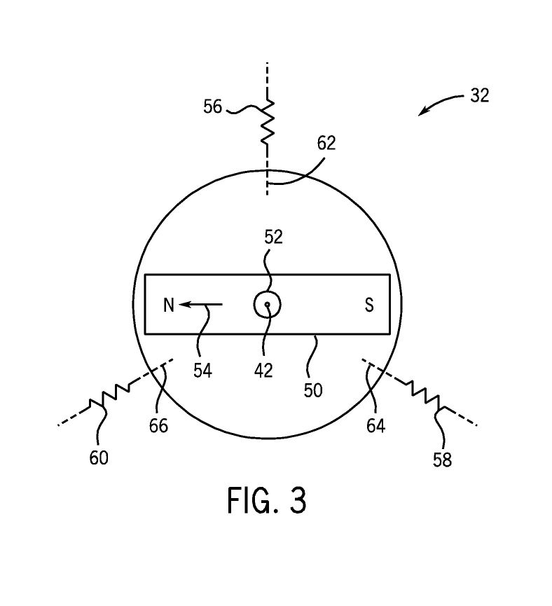

The motor 32 generally includes an electric motor. Specifically, an embodiment includes a three-phase synchronous motor 32, as discussed in further detail below with regard to FIG. 3. The motor 32 may be powered via wires 38. For example, the wire 38 may electrically couple the motor 32 to a power supply, such as the drive unit 16, a motor drive, a battery, or the like.

In an embodiment, the transducer 34 may include a device that is used to image the interior of the passage 36. For example, in medical application, the transducer 34 may include an ultrasonic transducer having a field of view 40. In one embodiment, information may be transmitted and received by the transducer 34 via the wires 38. For example, the wires 38 may couple the transducer 34 to the processor 18. During operation, rotating the transducer 34 about an axis 42 provides a two-dimensional image of a section 44 of the walls 46 of the passage 36. In the illustrated embodiment, rotation of the transducer 34 is provided via the motor 32 and a shaft 48. For example, the shaft 48 couples the motor 32 to the transducer 34, thus, operating the motor 32 causes the transducer 34 to rotate about the axis 42. Accordingly, during a medical procedure, the catheter 26 may be inserted into the passage 36, the motor 32 operated, and the transducer 34 rotated about the axis 42 to provide a two-dimensional image of the section 44 of the walls 46 of the passage 36.

To provide a more complete image of the interior of the passage 36, the transducer 34 may be moved such that various sections 44 of the walls 46 are imaged by the transducer 34. For example, the transducer 34 may continue to provide images as the catheter 26 is pushed or pulled through a blood vessel (e.g., the passage 36). Accordingly, the transducer 34 may provide a series of two-dimensional images corresponding to a plurality of sections 44. Often, it is desirable that the series of images are correlated to one another to provide a complete image of the surrounding tissue. However, when the position of the catheter 26 and the transducer 34 is not tracked, the exact locations of the images acquired from the transducer 34 may be unknown. In other words, the series of images may be provided to the clinician or to the processor 18, but it may be difficult to determine how the images relate to one another, e.g., do they overlap or is there a gap between images. Accordingly, it may be exceedingly difficult to stitch the images together to provide a complete image of the passage 36.

In an embodiment of the present technique, the tracking system 10 senses the magnetic field generated by the motor 32 to enable tracking of the position of the motor 32. The tracked position of the motor 32 may be used determine the positions of transducer 34, and the tip 30 of the catheter 26. In other words, the relative positions of the motor 32, the transducer 34, and the catheter 26 is known such that the position of the motor 32 may be correlated to the positions of the other components.

Turning now to FIG. 3, an embodiment of the motor 32 in accordance with an aspect of the present technique is illustrated. The illustrated embodiment depicts a three-phase synchronous motor 32 having a rotor 50, drive shaft 52, and stator coils 56, 58, and 60 having axes 62, 64, and 66. The rotor 50 generally includes a magnetic field that is conducive to generating a torque when the stator coils 56, 58, and 60 are energized. In one embodiment, the rotor 50 includes a permanent magnet. The rotor 50 may be formed from iron, cobalt, nickel, or the like, for instance. As illustrated, the rotor 50 has a magnetic moment vector in the direction of arrow 54. Thus, the magnetic field rotates as the drive shaft 52 rotates. Further, in an embodiment that includes a relatively small rotor 50 (e.g., approximately a few millimeters in size or less), the rotor 50 has an approximately dipole magnetic field with the magnetic moment along the length of the rotor 50 and generally perpendicular to the rotational axis 42 of the drive shaft 52 (e.g., in the direction of arrow 54). Accordingly, the dipole magnetic field rotates as the drive shaft 52 rotates.

A rotating dipole magnetic field is equivalent to two collocated orthogonal dipole coils driven by a sinewave current and a cosinewave current, respectively. In other words, where two coils are collocated (e.g., have a common origin) and are oriented orthogonal (e.g., perpendicular) to one another, the resulting magnetic field moment generated by the two dipole coils includes a constant magnitude vector that rotates in a single plane that is orthogonal to both coils. Thus, the rotation of the rotor 50 having a dipole magnetic field with a constant magnitude about the rotational axis 42 generates a rotating magnetic field moment vector that behaves in a similar manner to the resulting magnetic moment vector of two collocated orthogonal dipole coils driven by a sinewave and a cosinewave current, respectively. The rotating dipole magnetic field of the magnetic rotor 50 can be tracked by the same method as two collocated orthogonal dipole coils are tracked.

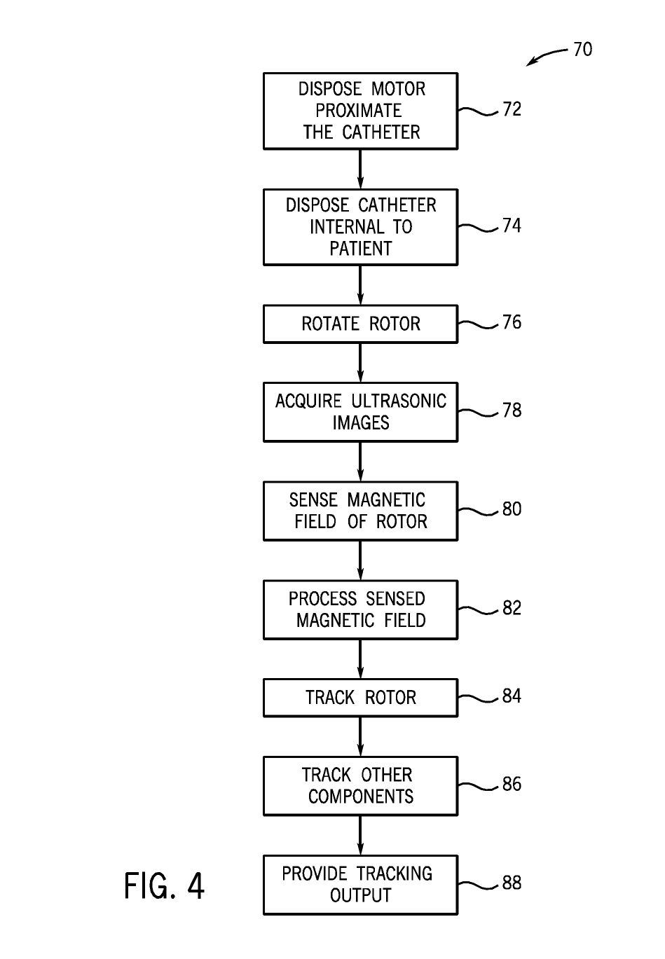

FIG. 4 illustrates a flow diagram of a method 70 including tracking a motor and other components (e.g., catheter 26 and transducer 34) based on the magnetic field of the rotor 50. The method 70 includes disposing the motor proximate the catheter, as illustrated at block 72. For example, in one embodiment, the motor 32 is disposed internal to the tip 30 of the catheter 26. In another embodiment, the motor 32 may be disposed in various locations internal to the body 28 of the catheter 26, and/or coupled to the exterior of the catheter 26.

The method 70 also includes, disposing the catheter internal to the patient, as illustrated at block 74. For example, the catheter 26 and the motor 32 may be threaded into the passage 36 such as a blood vessel, vein, artery, heart, or other cavity of the patient.

The method 70 includes rotating the rotor, as illustrated at block 76. For example, in one embodiment, rotating the rotor (block 76) includes providing power to the motor 32 to energize the stator coils 56, 58, and 60 to create a torque that induces rotation of the rotor 50. Rotating the rotor 50 may also provide for rotation of the transducer 34, as discussed above. In other words, the general operation of the motor 32 may be used to generate a rotating magnetic field via rotation of the rotor 50. Further, the step of rotating the rotor (block 76) may be performed prior to or after the step of disposing the catheter internal to the patient (block 74). For example, the motor 32 may be energized before the catheter 26 is inserted into the patient, or the motor 32 may be energized subsequent to the catheter 26 being disposed in the patient.

Further, the method 70 includes the step of acquiring ultrasonic images, as illustrated at block 78. As discussed above, acquiring ultrasonic images (block 78) may include imaging the passage walls 46 within the field of view 40 as the transducer 34 is rotated about the axis 42. Further, embodiments may include pulling the catheter 50 though the passage 36 as the motor 32 and the transducer 34 are rotated, enabling an increased area to be imaged.

Further, the tracking method 70 includes sensing the magnetic field of the rotor, as illustrated at block 80. For example, as the rotor 50 rotates the at least one receivers 14 of the tracking system 10 may sense the rotating magnetic field and pass a signal indicative of the sensed magnetic field to the processor 18. The step of sensing the magnetic field of the rotor (block 80) may be done at any time the rotor 50 is rotated, including, prior to, during, and after the step of acquiring ultrasonic images (block 78) and/or prior to, during, and after the catheter is disposed internal to the patient (block 74).

The method 70 also includes processing the sensed magnetic field, as illustrated at block 82. For example processing the sensed magnetic field may include the processor 18 implementing known tracking algorithms for tracking a two collocated orthogonal dipole coils driven by a sinewave and a cosinewave current, respectively. For example, processing may resolve the position (X, Y, Z) as well as the orientation (pitch, yaw and roll) of the rotor 50. Accordingly, processing may be implemented to track the rotor, as illustrated at block 84. Further, the tracked position of the rotor (block 84) may be used to track other components, as illustrated at block 86. For example, with the rotational path of the rotor 50 fixed relative to the catheter 26, and the transducer 34, the resolved position and orientation of the rotor 50 may be correlated to the position and orientation of the catheter 26, and the position and orientation of the transducer 34.

Further, the method 70 includes providing the tracking output, as illustrated at block 88. In one embodiment, the tracking output may include providing an output image indicating the position of the rotor 50, or other associated devices, overlaid on the image. Further, the tracking output may be used to correlate the acquired ultrasonic images (block 78) to a given position and orientation. Accordingly, a single image may be reconstructed that stitches together a plurality of images (e.g., fields of view 40 and sections 44) to generate a single reconstructed image that is output to the clinician or a corresponding data file (e.g., an image file or database).

Returning now to FIG. 3, the illustrated embodiment includes a first stator coil 86, a second stator coil 88, and a third stator coil 90 that surround the rotor 50. As will be appreciated, in the electric motor 32, each of the stator coils 56, 58, and 60 are provided a specific phase current that energizes the respective stator coil 56, 58, and 60. The combined current phases in the stator coils 56, 58, and 60 induce a torque to the rotor 50. Specifically, the illustrated embodiment includes a three-phase synchronous motor 32 including the rotor 50 (e.g., permanent magnet), the first stator coil 56, the second stator coil 58, and the third stator coil 60. Each of the stator coils 56, 58, and 60 generally includes a conductive winding about axes 62, 64, and 66 that are generally perpendicular to the drive shaft 52 and the rotational axis 42. For example, the first stator coil 86, the second stator coil 88, and the third stator coil 90 are each oriented to generate a magnetic field with a moment vector along a first axis 62, a second axis 64 and a third axis 66, respectively.

As will be appreciated, the three-phase synchronous motor 32 operates by driving a current across each of the stator coils 56, 58, and 60 via a three-phase power source. Specifically, the first stator coil 56 is driven by a current at a first phase, the second stator coil 58 is driven by a current at a second phase, and the third stator coil 60 is driven by a current at a third phase. Generally, the magnitude and frequency of the currents driving each of the coils is the same. In a three-phase power system the first-phase current is used as a reference, the second-phase current is delayed in time by one-third of the period of the electrical current, and the third-phase current is delayed by two-thirds of the period of the electrical current. Accordingly, in operation of the motor 32, the resulting moment of the stator coils 56, 58, and 60 creates a rotating magnetic field that provides the torque necessary to rotate the rotor 50. It will be appreciated that the frequency of the power supplied may be about 50 Hz in Europe and about 60 Hz in the United States.

In one embodiment, the tracking system 10 employs the magnetic fields generated by the stator coils 56, 58, and 60 as the transmitter 12 to enable tracking of the motor 32, the catheter 26, and/or the transducer 34. For example, in one embodiment, the processor 18 is coupled to the three-phase power source (e.g., drive unit 16) and, thus, may track each stator coil 56, 58, and 60. In other words, by knowing the phase of the currents driving each of the stator coils 56, 58, and 60, processing may demodulate the combined signals received from the at least one receiver 14 to determine the respective mutual inductance and, thus, the position of each stator coil 56, 58, and 60.

In another embodiment, the tracking system 10 may drive at least one additional current across the stator coils 56, 58, and 60 to induce at least one additional magnetic field that can be received and processed to track the position of the stator coils 56, 58, and 60 and the motor 32. For example, a high frequency current (e.g., 10 kHz or higher) may be driven across at least one stator coil 56, 58, and 60 to generate a magnetic field having the same high frequency. In one embodiment, the high frequency currents are too high in frequency for the rotor 50 to respond and, thus, have a minimal impact, if any, on the performance of the motor 32. For example, one embodiment includes driving a current with a frequency in the range of about 14 kHz to 20 kHz. Accordingly, the high frequency magnetic field may be sensed by the at least one receiver 14, and the signal processed to resolve the position and/or orientation of the stator coils 56, 58, and 60. In one embodiment, the high frequency (e.g., 14 kHz to 20 kHz) current may be driven across only one of the stator coils 56, 58, and 60. In another embodiment, the high frequency current may be driven across more than one of the stator coils 56, 58, and 60. For example, each of the coils may be driven by the same high frequency to generate a magnetic field at the given frequency. Further, an embodiment may include driving a different frequency across each of the stator coils 56, 58, and 60. In other words, the phase and/or frequency of the current driven across the stator coils 56, 58, and 60 may be varied such that each stator coils 56, 58, and 60 generates a unique magnetic field (e.g., in the range of about 14 kHz to 20 kHz). Accordingly, the varied magnetic field properties may enable the processor 18 to distinguish each of the magnetic fields.

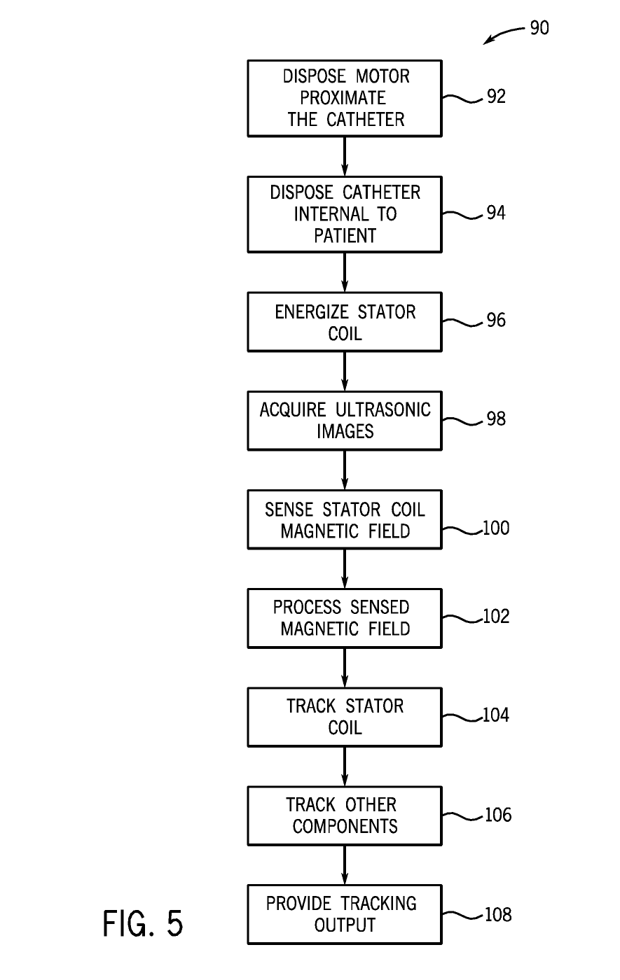

FIG. 5 illustrates a flow diagram of a method 90 including tracking the motor 32 and other components (e.g., catheter 26 and transducer 34) based on the magnetic field generated by the stator coils 56, 58, and 60 of the motor 32. The method 90 includes disposing the motor proximate the catheter, as illustrated at block 92. For example, in one embodiment, the motor 32 is disposed internal to the catheter tip 30. In another embodiment, the motor 32 may be disposed in various locations internal to the body 28 of the catheter 26, and/or coupled to the exterior of the catheter 26.

The method 90 also includes, disposing the catheter internal to the patient, as illustrated at block 94. For example, the catheter 26 including the motor 32 may be threaded into a passage 36 such as a blood vessel, vein, artery, heart, or other cavity of the patient.

The method 90 includes energizing a stator coil, as illustrated at block 96. For example, in one embodiment, energizing a stator coil (block 96) includes providing power (e.g., three-phase power) to the motor 32 to energize the stator coils 56, 58, and 60 and generate a torque that rotates the rotor 50. In another embodiment, energizing the stator coil (block 96) may include driving a current across one or a plurality of the stator coils 56, 58, and 60. For example, a single high frequency may be driven across one of the stator coils 56, 58, and 60, or a plurality of the stator coils 56, 58, and 60 may be driven by various high frequency currents. For example, a current source (e.g., drive unit 16 or current source 22) may drive a high frequency current across at least one of the stator coils 56, 58, and 60 of the motor 32 (block 96). The high frequency may be of a sufficiently high frequency that it does not affect operation of the motor 32, and may be driven across the coil whether or not the motor 32 is being operated by an operating current (e.g., three-phase power). The step of energizing the stator coil (block 96) may be performed prior to or after the step of disposing the catheter internal to the patient (block 94). For example, the stator coils 56, 58, and 60 may be energized before the catheter 26 is inserted into the patient, or the stator coils 56, 58, and 60 may be energized subsequent to the catheter 26 being disposed in the patient.

Further, method 90 includes a step of acquiring ultrasonic images at block 98. As discussed above, acquiring ultrasonic images may include imaging the passage walls 46 with the field of view 40 of the transducer 34. For example, the transducer 34 (e.g., ultrasonic) may be rotated about the axis 42 to acquire a series of two-dimensional images. Further, embodiments may include pulling the catheter 26 though the passage 36 to enable an increased area to be imaged.

The method 90 also includes sensing the stator core magnetic field, as illustrated at block 100. For example, in one embodiment, the at least one receiver 14 of the tracking system 10 may sense the magnetic field generated by at least one of the stator coils 56, 58, and 60 as a result of the current used to operate the motor 32. Thus, sensing the stator coil magnetic field (block 100) may include sensing at least one magnetic field indicative of at least one phase of the three phases of power used to operate the motor 32. In another embodiment, the at least on receiver 14 may be employed to sense a specific magnetic field driven across the at least one of the stator coils 56, 58, and 60. For instance, the at least one receiver 14 may sense the high frequency magnetic field generated at block 96, and pass a signal indicative of the high frequency magnetic field to the processor 18 for processing.

The method 90 also includes processing the sensed magnetic field, as illustrated at block 102. For example processing the sensed magnetic field (block 102) may include the processor 18 implementing known tracking algorithms for tracking a single or multiple coil transmitters 12. For example, where each of the coils is driven out of phase, the processor may implement algorithms to demodulate each respective phase to track the position of each coil. Further, processing may use the relative locations of each of the fixed stator coils 56, 58, and 60 to resolve additional degrees of freedom. For example, processing may resolve the position (X, Y, Z) as well as the orientation (pitch, yaw and roll) of the stator coils 56, 58, and 60. A similar processing technique may be applied where each of the stator coils 56, 58, and 60 are driven at varying high frequencies that can be processed individually. Further, in an embodiment, a single stator coil 56, 58, and 60 may be driven by a high frequency current and, thus, processing may employ the above discussed processing techniques for a single dipole coil. Accordingly, processing may be implemented to track the stator coils, as illustrated at block 104. Further, the tracked position of the stator coils (block 104) may be used to track other components, as illustrated at block 106. For example, with the position of the stator coils 56, 58, and 60 fixed relative to the catheter 26, and the transducer 34, the resolved position and orientation of the stator coils 56, 58, and 60 may be correlated to the position and orientation of the catheter 26 and the transducer 34.

The method 90 also includes providing the tracking output, as illustrated at block 108. In one embodiment, the tracking output may include providing an output image with the position of the stator coils 56, 58, and 60, or other associated devices, overlaid on the image. Further, the tracking output may be used to correlate the acquired ultrasonic images (block 98) to a given position and orientation. Accordingly, a single image may be reconstructed having a plurality of images (e.g., sections 44) stitched together to generate a single reconstructed image that is output to the clinician or a corresponding file.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A tracking system, comprising:
a motor coupled to a medical instrument and configured to generate at least one magnetic field; and
at least one receiver coil configured to sense the magnetic field.

2. The tracking system of claim 1, comprising a drive unit configured to drive a high frequency current across at least one stator coil of the motor to generate the at least one magnetic field.

3. The tracking system of claim 1, wherein the motor comprises a permanent magnet rotor.

4. The tracking system of claim 3, wherein permanent magnet rotor comprises an approximately dipole magnetic field.

5. The tracking system of claim 3, wherein rotating the permanent magnet rotor generates a rotating dipole magnetic field.

6. The tracking system of claim 1, wherein the motor comprises a three-phase synchronous motor.

7. The tracking system of claim 1, comprising a plurality of the at least one receiver coils.

8. The tracking system of claim 1, comprising a processor configured to determine a position of the motor based on the magnetic field.

9. A method of tracking, comprising:
rotating a rotor of a motor, wherein the rotor comprises a permanent magnet that generates a rotating magnetic field when the rotor is rotated;
sensing the rotating magnetic field with at least one receiver;
transmitting to a processor a signal indicative of the rotating magnetic field; and
processing the signal to determine a position of the motor.

10. The method of claim 9, comprising tracking the position of the motor based on the determined position.

11. The method of claim 9, comprising displaying an image indicative of the position of the motor.

12. The method of claim 9, comprising disposing a catheter internal to a patient, wherein the catheter is coupled to the motor.

13. The method of claim 12, comprising tracking the position of the catheter based on the position of the motor.

14. The method of claim 9, comprising imaging an internal region of a patient.

15. The method of claim 14, comprising associating an image of the internal region to an image location based on the determined position of the motor.

16. The method of claim 15, comprising acquiring a plurality of the images and combining the plurality of the images into a single combined image based on the image locations.

17. A method of tracking, comprising:
energizing a stator coil of a motor to generate at least one magnetic field;
sensing the at least one magnetic field with at least one receiver;
transmitting to a processor a signal indicative of the at least one magnetic field; and
processing the signal to determine a position of the motor.

18. The method of claim 17, wherein energizing the stator coil comprises providing three-phase power to each stator of the motor.

19. The method of claim 18, wherein energizing the stator coil comprises inducing a high frequency current across at least one stator of the motor.

20. The method of claim 17, comprising tracking the position of the motor based on the determined position.

21. The method of claim 17, comprising displaying an image indicative of the position of the motor.

22. The method of claim 17, comprising disposing a catheter internal to a patient, wherein the catheter is coupled to the motor.

23. The method of claim 22, comprising tracking the position of the catheter based on the position of the motor.

24. The method of claim 17, comprising imaging an internal region of a patient.

25. The method of claim 24, comprising associating an image of the internal region to an image position based on the determined position of the motor.
